(19) United States
(12) Patent Application Publication
(10) Pub. No.: US 2026/0307105 A1
(43) Pub. Date: Oct. 8, 2026

(54) CMUT DRIVE METHOD

(71) Applicant: XIVER MEMS Foundry B.V., Eindhoven (NL)

(72) Inventors: Willem-Jan Arend DE WIJS, Eindhoven (NL); Johan Hendrik KLOOTWIJK, Eindhoven (NL); Hans-Peter LOEBL, Eindhoven (NL)

(73) Assignee: XIVER MEMS Foundry B.V., Eindhoven (NL)

(21) Appl. No.: 19/116,094

(22) PCT Filed: Sep. 20, 2023
(86) PCT No.: PCT/EP2023/075873

§ 371 Date: Mar. 27, 2025

(30) Foreign Application Priority Data
Sep. 27, 2022 (EP) 22197910.7

(51) Int. Cl. B06B 1/02; A61B 8/00

(52) U.S. Cl. CPC B06B 1/0215; A61B 8/4494; A61B 8/54; B06B 1/0292; B06B 2201/76

(57) ABSTRACT

A method for improving receive sensitivity of a cMUT transducer element by dynamically adjusting the bias volage between the transmit and receive phases of the drive cycle while keeping the cMUT in collapsed operation mode at all times.

### FIELD OF THE INVENTION

The present invention relates to a drive method for cMUT cells, particularly in the context of harmonic ultrasound imaging.

### BACKGROUND OF THE INVENTION

Capacitive micromachined ultrasonic transducers (cMUTs) generally combine mechanical and electronic components in very small packages. The mechanical and electronic components operate together to transform mechanical energy into electrical energy and vice versa. Because cMUTs are typically very small and have both mechanical and electrical parts, they are commonly referred to as micro-electronic mechanical systems (“MEMS”) devices. cMUTs, due to their small size, can be used in numerous applications in many different technical fields, including medical device technology.

One application for cMUTs within the medical device field is imaging soft tissue. Tissue harmonic imaging has become important in medical ultrasound imaging because it provides unique information about the imaged tissue. In harmonic imaging, ultrasonic energy is transmitted from an imaging array to tissue at a center frequency (f0) during transmission. This ultrasonic energy interacts with the tissue in a nonlinear fashion, especially at high amplitude levels, and ultrasound energy at higher harmonics of the input frequency, such as 2f0, are generated. These harmonic signals are then received by the imaging array, and an image is formed. To achieve a good signal-to-noise ratio during harmonic imaging, ultrasonic transducers in the imaging array would preferably be sensitive around both the fundamental frequency f0 and the first harmonic frequency 2fo.

Conventional ultrasonic transducers are not capable of performing in such a manner. For example, piezoelectric transducers are not suitable for harmonic imaging applications because these transducers tend to be efficient only at a fundamental frequency (f0) and its odd harmonics (3f0, 5f0, etc.). To compensate for the odd harmonic efficiencies of piezoelectric transducers, the transducer is typically damped, and several matching layers are used to create a broad band (~90% fractional bandwidth) transducer. This approach, however, requires a trade-off between sensitivity and bandwidth since significant energy is lost due to the backing and matching layers. Additionally, conventional piezoelectric transducers and fabrication methods do not enable device manufacturers to control or adjust the vibration harmonics of conventional piezoelectric transducers.

cMUT transducers are suitable for use for the purpose of harmonic imaging. These can be operated to utilize multiple vibration modes of the cMUT membrane and permit adjustable vibration modes and/or controllable vibration harmonics. Harmonic imaging cMUTs are designed to achieve higher sensitivity over a wide bandwidth and adapted to exploit multiple vibration modes of a cMUT membrane.

Thus, for harmonic mode imaging, the excitation vibration of the cMUT is at a lower frequency than in the receive mode, since in the receive mode, the cMUT samples a multiple of the frequency in the excitation mode, for image formation. This means that an increase in receive sensitivity at higher frequencies should also lead to an increased performance.

It is known that increasing the bias of a cMUT element increases the sensitivity for higher frequencies.

However, there is a maximum field, and corresponding maximum voltage that can be applied, above which the dielectric layers in the device break down. This is known as the breakdown voltage. The combination of the bias voltage and the RF voltage must not exceed this breakdown voltage. Indeed, in practice, the cMUT is typically operated well below the breakdown voltage to avoid tunnelling of electrons through the dielectric. For example, for a cMUT with breakdown field at ~7-9 MV/cm, a typical upper operating limit might be set at a voltage which corresponds to a field of around 4.5-5 MV/cm (where voltage=field*total thickness of the cMUT dielectric). A cMUT may typically include at least one dielectric layer between the lower electrode and the cMUT cavity and another dielectric layer between the upper electrode and the cavity. Breakdown voltage is dependent on the dielectric thickness, equal to the total thickness of all dielectric layers between the electrodes. The breakdown voltage may typically be in the range of 150-200 V, alternatively in the range of 70-100 V, alternatively in the range of 60-80 V.

U.S. Pat. No. 10,313,027 B2 discloses a wide band through-body communication system adapted to communicate data through the body ultrasonically, in which a cMUT transmitter operated in a collapsed mode is configured to transmit ultrasonic data signals within a broad band of operating frequencies through the body to a similarly configured cMUT receiver for decoding and processing.

### SUMMARY OF THE INVENTION

The invention is defined by the claims.

According to examples in accordance with an aspect of the invention, there is provided a method for driving a cMUT device with a drive cycle comprising a transmit period and a receive period. The method comprises: in the transmit period, driving a cMUT element of the cMUT device with a first bias voltage and an RF voltage; and in the receive period, driving the cMUT element with a second bias voltage, and without an RF voltage. The second bias voltage is higher than the first bias voltage, and the combined RF voltage and the first bias voltage are such as to cause the cMUT element to operate in a collapsed mode during the transmit period. The second bias voltage is such as to cause the cMUT element to operate in collapsed mode during the receive period.

Thus, the concept proposed by the inventors is to vary the bias voltage between the transmit and receive cycle phases. After generation of the ultrasound wave in the transmit period, the bias voltage can be safely increased during the receive period due to the removal of the applied RF voltage. In this way, it is possible to improve sensitivity in receive period. The bias voltage can then be lowered again before the next generation of the ultrasound wave in the next transmit period. By driving the cMUT element with a second bias voltage higher than the first bias voltage in the receive period, the receive sensitivity of the cMUT element is higher at frequencies higher than the frequency of the RF voltage.

Thus, it is proposed to use a separation in time principle for adjusting the bias voltage level. Importantly however, it is proposed to configure the bias and RF voltage levels such the cMUT stays in collapse mode at all times, during both transmit and receive periods. This circumvents known problems in state-of-the-art technology, wherein artefacts can occur, and also reduces reaction time of the membranes, as the motion required is lower.

Working in the collapse mode is advantageous compared to non-collapse mode. The cMUT has higher transmit pressure in the collapse mode. To obtain high transmit pressure in non-collapse mode, it would be necessary to operate the cMUT at a bias voltage close to the collapse point. The resulting device would then show then a more non-linear behavior which, for purposes of harmonic imaging, is highly disadvantageous. Furthermore, switching between collapse and non-collapse mode leads to wear and reduces lifetime (reliability) of the cMUT. Acoustic artefacts can also occur when the membrane moves out of collapse and into collapse state due to the sudden change in capacitance, which translates into artefacts in the signals that need to be filtered out. Therefore staying in collapse mode avoids the need to filter these artefacts and increases device lifetime.

The way of addressing the issue of receive sensitivity in the state of the art is to use dynamic gain control, where the level of amplification of the signal is adjusted as a function of time in the receive phase of the ultrasound probe. Embodiments of the present invention thus provide an additional and/or alternative way to increase the receive signal, prior to any amplification. This therefore provides a new way to improve performance. Moreover, boosting the receive signal prior to amplification is also beneficial for signal-to-noise-ratio (SNR).

As discussed, the proposed method finds particularly advantageous application for harmonic ultrasound imaging, for example using a cMUT adapted for harmonic ultrasound imaging. The drive cycle may be a harmonic imaging cycle. However, the general principle can be applied to a cMUT of any type for harmonic or non-harmonic imaging since in all cases an increase in receive sensitivity is achieved,

The cMUT device comprises one or more cMUT transducer elements.

For avoidance of doubt, in the context of the present application, the RF voltage means an alternating voltage. The bias voltage means a DC voltage.

The sum of the RF voltage and the first bias voltage, and separately the second bias voltage, should each at all times not exceed a pre-defined maximum voltage, representing a breakdown voltage of the cMUT element. In practice, the voltages in the two modes may be configured such that they are kept below an upper limit which is a defined margin below the breakdown voltage. For example, for a cMUT with a breakdown field of ~7-9 MV/cm, a typical upper operating limit might be set at a voltage corresponding to a field of around 4.5-5 MV/cm (where breakdown field [V/cm]=breakdown voltage/total thickness of the dielectric layers of the cMUT element). Thus, preferably the maximum voltage in both transmit and receive periods is kept below a pre-defined upper limit which is lower than the breakdown voltage and which is chosen as a manufacturing choice depending on the lifetime requirements of the cMUT device.

The cMUT operates in collapse mode when the voltage applied to it (the combined first bias and RF voltage, or the second bias) exceeds the collapse voltage for the cMUT element. In preferred embodiments, the bias voltage in both the transmit and receive periods is set above the collapse voltage. This is beneficial for the lifetime of the cMUT transducer.

According to some embodiments, the difference between the second bias voltage and the first bias voltage may be equal to a voltage amplitude of the RF voltage. This means that the step up in the bias voltage exactly matches the size of the RF voltage. If for example the RF+bias in the transmit mode is at or close to the maximum operational voltage, then this feature ensures that the maximum possible bias voltage increase is attained in the receive mode without exceeding the maximum operation voltage.

In some embodiments, the method further comprises sampling the cMUT element during the receive period to obtain a receive signal.

In some embodiments, a transition from the transmit period to the receive period of the imaging cycle comprises a ramp-up of the first bias voltage to the second bias voltage, according to a first ramping function. In some embodiments, a transition from the receive period to the transmit period of the cycle comprises a ramp-down of the second bias voltage to the first bias voltage according to a second ramping function.

In some embodiments, the first and second ramping function are controllable.

In some embodiments, each of the first and second ramping functions is a smooth linear function.

In some embodiments, the method further comprises sampling the cMUT element during the receive period to obtain a receive signal, and wherein said sampling comprises sampling only between the end of the ramp-up of the bias voltage and the beginning of the ramp-down of the bias voltage.

In some embodiments, the method further comprises obtaining an indication of one or more target acoustic frequencies to be sampled during the receive period and determining a value of the second bias voltage in dependence upon the one or more target acoustic frequencies. In other words, it is proposed according to this set of embodiments to tune the bias voltage in accordance with a harmonic frequency to be measured. This may for example make use of a pre-defined mapping function or lookup table which relates target frequencies to optimal bias voltages for sampling those frequencies.

In some embodiments, the method comprises determining the one or more target acoustic frequencies, and wherein the one or more target acoustic frequencies are each harmonics of the frequency of the RF voltage applied during the transmit period. In some embodiments, one or more target acoustic frequencies include a third harmonic of the frequency of the RF voltage applied during the transmit period.

The invention can also be embodied in hardware form.

In particular, another aspect of the invention is a cMUT apparatus, comprising: a cMUT element; and drive electronics, adapted to drive a cMUT device with a drive cycle comprising a transmit period and a receive period. The drive electronics are adapted to: in the transmit period, drive the cMUT element with a first bias voltage and an RF voltage; in the receive period, drive the cMUT element with a second bias voltage, and without an RF voltage; wherein the second bias voltage is higher than the first bias voltage; and wherein the combined RF voltage and first bias voltage cause the cMUT element to operate in a collapsed mode during the transmit period, and wherein the second bias voltage causes the cMUT element to operate in collapsed mode during the receive period.

The apparatus may further comprise signal sampling electronics adapted to sample the cMUT element during the receive period to obtain a receive signal.

In some embodiments, a transition from the transmit period to the receive period of the imaging cycle comprises a ramp-up of the first bias voltage to the second bias voltage, according to a first ramping function; and wherein a transition from the receive period to the transmit period of the cycle comprises a ramp-down of the second bias voltage to the first bias voltage according to a second ramping function.

In particular, further aspects of the invention relate to an ultrasound probe, comprising the cMUT apparatus as defined above, and to an ultrasound imaging system, comprising such ultrasound probe.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows the structure of a cMUT element;

FIG. 2 shows the example cMUT element operated in collapse mode;

FIG. 3 outlines steps of an example method in accordance with one or more embodiments of the invention;

FIG. 4 shows components of an example apparatus in accordance with one or more embodiments;

FIGS. 5-8 illustrate example voltage signal characteristics during transmit and receive periods according to one or more embodiments; and

FIG. 9 shows an example ultrasound imaging system with receive or sampling electronics and drive electronics.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a method for improving receive sensitivity of a cMUT transducer element by dynamically adjusting the bias volage between the transmit and receive phases of the drive cycle while keeping the cMUT in collapsed operation mode at all times. The bias voltage is increased in receive mode to increase sensitivity.

To aid understanding, some brief background explanation will be provided regarding collapse mode operation of cMUT elements.

As described in the paper Micromachined Ultrasonic Transducers, IEEE Trans UFFC, Vol. 50, No. 9(2003), for a conventional capacitive micromachined ultrasonic transducer (cMUT) to be operated in collapsed mode, the flexible membrane of the cMUT is typically excited with a voltage that causes part of the membrane to collapse onto the corresponding cMUT substrate. Subsequent reduction of the voltage applied to the membrane to a certain threshold voltage, commonly characterized as the cMUT ‘snapback voltage’, will typically cause the membrane to lift upward from the substrate, and to return to an equilibrium position. By contrast, to the extent the voltage applied to a previously collapsed membrane is kept above the snapback voltage, a fairly linear and efficient output of the device typically can be achieved.

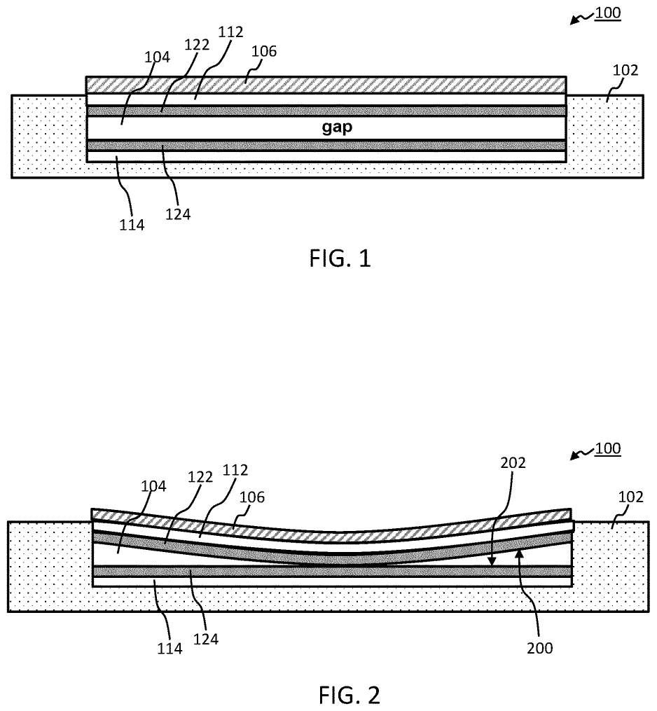

A conventional cMUT structure is shown in FIG. 1. More particularly, FIG. 1 shows a cMUT 100 in schematic cross section including a substrate 102 in which a pocket or cavity 104 is formed, and a flexible membrane 106 mounted to the substrate 102 across the cavity 104. A first electrode 112 is positioned atop the membrane 106, and a second electrode 114 is positioned beneath the cavity. A first dielectric layer 122 may be disposed between the first electrode 112 and the cavity 104. A second dielectric layer 124 is disposed between the second electrode 114 and the cavity 104. This therefore forms an upper layer stack suspended above the cavity 104 comprising the membrane 106 disposed atop the first electrode 112, disposed atop the first dielectric layer 122 and a second layer stack at a base of the cavity 104 comprising the second dielectric layer 124 disposed atop the second electrode 114.

The total dielectric thickness of the cMUT is equal to the sum of the thicknesses of the dielectric layers. The breakdown voltage means the voltage which leads to breakdown of these dielectric layers. At the breakdown voltage point, current starts to flow from electrode 112 to 114, effectively destroying the capacitor structure and resulting in the structure behaving as a resistor. As a result, the device heats up quickly and can burn through.

In circumstances in which the bias voltage applied between the electrodes is set at a relatively low voltage, or at zero volts, the cMUT 100 will typically exhibit a gap within the cavity 104 between the flexible membrane 106 and the substrate 102.

Referring now to FIG. 2, in operation, upon a voltage bias applied between the first 112 and second 114 electrode being increased a sufficient amount from the relatively low or zero level associated with the configuration of the cMUT 100 shown in FIG. 1, the flexible membrane 106 will tend to collapse downward into the cavity 104 and toward the substrate 102. Such collapse of the flexible membrane 106 can substantially eliminate the gap (FIG. 1) between the flexible membrane 106 and the substrate 102, such that a downward-facing surface 200 of the upper layer stack 106, 112, 122 is at least temporarily placed in physical contact with a corresponding upward-facing surface 202 of the lower layer stack 114, 124. This collapsed condition of the flexible membrane 106 with respect the substrate 102, once achieved, may be maintained by the continuous application across the flexible membrane 106 and the substrate 102 of a voltage in excess of a certain minimum level, commonly referred to as the collapse voltage or the snapback voltage.

Embodiments of the present invention have particularly powerful application within the context of harmonic ultrasound imaging. As explained earlier in this document, for harmonic ultrasound imaging, it is necessary for the cMUT transducer to sense echo waves at multiples of the transmit frequency, i.e. higher harmonics of the transmit frequency. This requires high sensitivity to higher frequencies. Collapsed mode operation (by maintaining the bias voltage above the collapse voltage) increases the sensitivity of the cMUT to higher frequencies without the need to increase the bias voltage too close to operational limits. In other words, in order to achieve higher receive sensitivity in non-collapse made, it is necessary to operate the cMUT in a regime close to the dielectric breakdown voltage, which leads to a more non-linear receive behavior, which is less optimal particularly for harmonic imaging.

Embodiments of the present invention facilitate the provision of harmonic imaging operation with improved receive sensitivity at the harmonic frequencies of the central base frequency.

Embodiments of the invention are based on the insight of using time separation for adjusting the bias voltage level of the cMUT element(s) comprised in the cMUT device during the transmit/receive drive cycle, so as to provide a dynamic bias voltage control, in which, after the generation of the transmit ultrasound wave, the bias voltage is increased with the value of the RF voltage applied, in order to improve sensitivity in the receive phase for the higher (harmonic) frequencies, and then lowered again before the next transmit event. During the entire cycle, the cMUT element(s) stays in collapse mode at all times.

Embodiments of the invention are based on the insight that the bias voltage can be safely increased during the receive phase because the additional RF voltage applied during the transmit phase is not needed during the receive phase. This leaves scope to increase the bias voltage without risking exceeding the safe upper operating limit for total applied voltage, which is typically some fraction, e.g. between 50-80% of the breakdown voltage of the transducer.

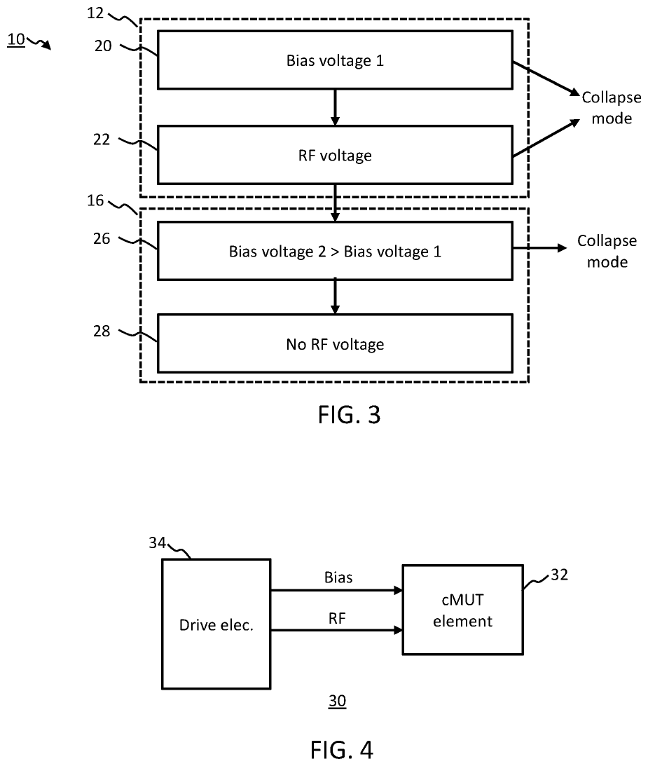

FIG. 3 outlines in block diagram form steps of an example method according to one or more embodiments. The steps will be recited in summary, before being explained further in the form of example embodiments.

Provided is a method 10 for driving a cMUT device comprising one or more cMUT elements with a drive cycle comprising a transmit period 12 and a receive period 16. The method comprises, in the transmit period 12, driving a cMUT element of the cMUT device with a first bias voltage 20 and an RF voltage 22. The method comprises, in the receive period 16, driving the cMUT element with a second bias voltage 26, and without an RF voltage 28. The second bias voltage 26 is higher than the first bias voltage 20. The combined RF voltage 22 and the first bias voltage 20 are such as to cause the cMUT element to operate in a collapsed mode (at all times) during the transmit period, and the second bias voltage 26 is such as to cause the cMUT element to operate in collapsed mode during the receive period.

As noted above, the method can also be embodied in hardware form.

With reference to FIG. 4, another aspect of the invention is a cMUT apparatus 30. The cMUT apparatus comprises a cMUT device comprising at least one cMUT element 32. The apparatus further comprises drive electronics 34, adapted to drive a cMUT element with a drive cycle comprising a transmit period and a receive period. The drive electronics are adapted to: in the transmit period, drive the cMUT element with a first bias voltage and an RF voltage; in the receive period, drive the cMUT element with a second bias voltage, and without an RF voltage; wherein the second bias voltage is higher than the first bias voltage; and wherein the combined RF voltage and first bias voltage cause the cMUT element to operate in a collapsed mode during the transmit period, and wherein the second bias voltage causes the cMUT element to operate in collapsed mode during the receive period.

With regards to the drive electronics, these are arranged to drive the at least one cMUT transducer element (either directly or via a microbeamformer) during the transmission mode. The drive electronics may further include a transmit/receive (T/R) switch for switching the at least one cMUT element from transmit to receive mode.

In some embodiments, the apparatus may further comprise signal sampling electronics adapted to sample the cMUT element during the receive period to obtain a receive signal.

With regards to the cMUT element itself, the structure of such an element is well known and has already been described above with reference to FIG. 1 and FIG. 2. For operation, additionally a pair of electrodes is provided, one applied to the membrane and another coupled atop the substrate, or just beneath the cavity. In transmit mode, the bias and the RF voltages are applied together across the two electrodes. In receive mode, the bias voltage is applied across the two electrodes.

Standard downstream processing electronics and software to generate an image may also be provided according to some embodiments. For example, a processing device may be provided configured to process the received signals from the cMUT to generate a harmonic image dataset.

An important feature of the proposed concept is to dynamically adjust the bias voltage between the transmit and receive phase, but in such a way that in both phases the cMUT is operating in collapse mode. Collapse mode operation will be known to the skilled person in this field, and it has already been described above with reference to FIG. 2.

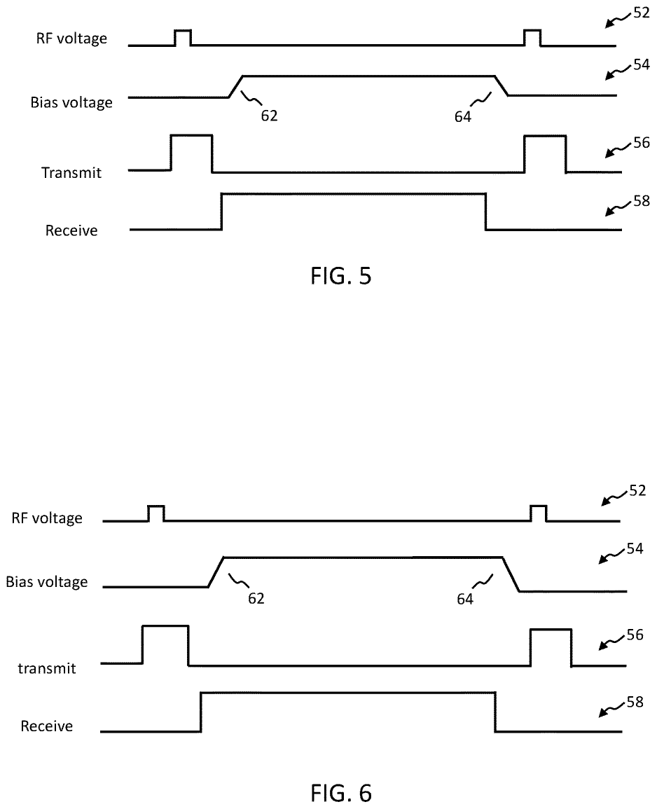

FIG. 5 illustrates voltage characteristics during transmit and receive periods of the drive cycle according to at least one set of embodiments of the present invention. The transmit and receive period timings respectively are indicated by duty cycle waveforms 56, 58 in FIG. 5.

As indicated, the bias voltage 54 is increased during the receive period, transitioning from a first bias voltage in the transmit period to a second (higher) bias voltage in the receive period. It is then decreased again to the first bias voltage once the receive period has ended, ready for the next transmit period.

The bias voltage in both the transmit and receive periods is such as to cause the cMUT elements to operate in collapse mode, i.e. it exceeds the collapse voltage for the cMUT. The collapse voltage can be easily identified for any cMUT since it is the minimum applied bias voltage at which the membrane switches into its collapsed state, as discussed above with reference to FIG. 2.

Preferably, and as illustrated in the example of FIG. 5, a transition from the transmit period to the receive period of the drive cycle comprises a ramp-up of the first bias voltage to the second bias voltage, according to a first ramping function 62. Preferably, a transition from the receive period to the transmit period of the drive cycle comprises a ramp-down of the second bias voltage to the first bias voltage according to a second ramping function 64. Using a ramping function rather than a step change avoids the change in bias voltage causing generation of a transmit pulse, which is not the intended effect.

With regards to particular values for the first bias voltage, second bias voltage and RF voltage, these can be configured according to preferences or requirements of the particular hardware and the particular application, so long as the constraints already discussed are met.

A further constraint which typically should be met is that the applied voltage(s) in both transmit and receive mode should not exceed an upper operation limit, which is usually chosen as some fraction, e.g. between 50-80%, of the breakdown voltage.

The breakdown voltage is a voltage above which the dielectric layers in the device break down. This voltage level can be readily tested for any cMUT element by incrementally increasing the applied voltage, V, while simultaneously monitoring current, I. Plotting an IV curve allows determination of the voltage value at which breakdown (destruction) of the device occurs. This is identifiable as a voltage point at which a sudden inflection point in the IV curve occurs. In particular, it can be identified as a voltage at which the current increases significantly (i.e. at a faster rate than in the preceding IV curve). Before breakdown occurs, the tunneling regime can also be identified.

The combination of the bias voltage and the RF voltage (if applied) must at all times not exceed this breakdown voltage. Since the RF voltage cycles between an upper and lower amplitude, it is more precise to say the sum of the maximum amplitude of the RF voltage plus the first bias voltage should not exceed the breakdown voltage, and the second bias voltage alone should not exceed the breakdown voltage.

In fact, in practice, it is preferable that the cMUT is operated well below the breakdown voltage to avoid tunnelling of electrons through the dielectric. For example, for a cMUT with breakdown voltage of 170-200 V, a typical upper operating limit might be set at around 150V-180V. However, the particular selection of the upper operating limit, as a fraction of the breakdown voltage, can be a manufacturing choice; it represents a balancing between device lifetime (lower upper voltage limit leads to longer lifetime) and device sensitivity (higher upper voltage level leads to increased sensitivity).

The frequency to which a cMUT element is sensitive during the receive mode is a function of the applied bias voltage, with increased bias voltage leading to an increase in the frequency to which the cMUT is sensitive in the receive mode.

For harmonic imaging, the imaging principle relies on sensing the higher harmonics of the transmit center frequency. Therefore, the higher the target harmonic, the higher must be the bias voltage in the receive mode relative to the bias voltage in the transmit mode; i.e. the higher must be the difference between the first and second bias voltages discussed above.

Following this logic, in some embodiments, to further increase the difference between the transmit frequency and the frequency to which the cMUT is sensitive during receive mode, the first bias voltage (during the transmit period) can be decreased further to increase the difference between transmit center frequency and the receive frequency sensitivity. This is illustrated in FIG. 6, which shows that the first bias voltage, during the transmit period is reduced compared to the example shown in FIG. 5. This therefore provides a way to increase the difference between the transmit and receive frequencies without further increasing the bias voltage in the receive mode (which might risk exceeding the maximum operating level). This however comes as the cost of slightly reducing the transmit pressure due to the lower bias voltage in the transmit period. Thus, this is an optional variable which can be optimized according to manufacturing preferences.

To illustrate the concept, and without limiting the general scope of the invention, by way of one example, a lower limit of a suitable cMUT device transmit center frequency might be about 1.8 MHz. This means that with increased receive sensitivity, the third harmonic of the center transmit frequency might be detected.

To illustrate further, an example might be considered in which the combination of the first bias voltage and the RF voltage during transmit phase is (at the maximum of the RF cycle) 180 Volts. Any combination of bias voltage level and RF voltage amplitude can be chosen, as long as the bias voltage level during the transmit phase is larger than the collapse voltage (e.g. in the order of around 60 volts).

For example, some illustrative combinations would, for this particular example being considered, include the following:
Transmit bias 140 V, RF 40 V, receive bias 180 V.
Transmit bias 80 V, RF 100V, receive bias 180 V.
Transmit bias 120 V, RF 40 V, receive bias 180 V.

By way of example, in the above cases, the breakdown voltage might be around 200V, so that the total applied voltage in all cases is some margin below the breakdown voltage.

Optionally, the difference between the second bias voltage and the first bias voltage might be equal to a (maximum) voltage amplitude of the RF voltage. This means that the step up in the bias voltage exactly matches the size of the RF voltage at the maximum point of its RF cycle. If for example the RF+bias in the transmit mode is at or close to the maximum operational voltage, then this feature ensures that the maximum possible bias voltage increase is attained in the receive mode without exceeding the maximum operation voltage.

As noted above, the transition from the transmit period to the receive period of the drive cycle can comprises a ramp-up of the first bias voltage to the second bias voltage, according to a first ramping function 62 and a transition from the receive period to the transmit period of the drive cycle can comprise a ramp-down of the second bias voltage to the first bias voltage according to a second ramping function 64.

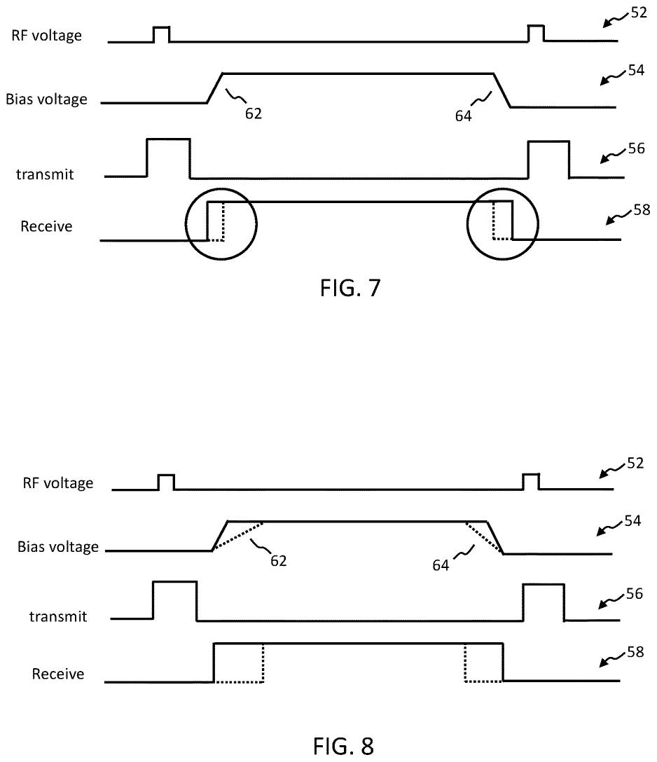

In some embodiments, and as illustrated in FIG. 7, the timing of the receive period 58 can be adjusted so that the receive period only begins once the ramp-up 62 has finished and the receive period ends before the ramp-down begins 64. This therefore avoids disturbances to the receive electronics which might be caused by the ramping phases. The adjusted timings of the receive period are indicated in the circled regions in FIG. 7. The solid lines indicate the timings of the receive period before adjustment is made, and the dotted lines indicate the proposed adjustment to the timing, so that the receive period starts after the ramp-up has finished and ends before the ramp-down starts.

In other words, in some embodiments, the method further comprises sampling the cMUT element during the receive period to obtain a receive signal, and wherein said sampling comprises sampling only between the end of the ramp-up of the bias voltage and the beginning of the ramp-down of the bias voltage.

Additionally, in some embodiments, the shape and timing of the ramp-up 62 and ramp-down 64 functions of the bias voltage change can be modified, as indicated in FIG. 8. A shallower ramp-up and ramp-down function (illustrated by the dotted lines in FIG. 8) might reduce electronic and ultrasonic effects. In particular, a steep ramp-up or ramp-down may cause transmission of ultrasound which is not the intended effect in adjusting the bias-voltage for the receive phase.

In other words, in some embodiments, the first 62 and second 64 ramping function are controllable.

In some embodiments, each of the first 62 and second 64 ramping functions is a smooth linear function. The slope or gradient of the ramp up and/or ramp down functions could be adjustable. Other shapes of function however can also be used.

As discussed above, in some embodiments, the method further comprises sampling the cMUT element during the receive period to obtain a receive signal.

One particularly advantageous application for embodiments of the invention is for harmonic imaging.

To optimize the method and apparatus for harmonic imaging, in some embodiments, the method may optionally further comprise obtaining an indication of one or more target acoustic frequencies to be sampled during the receive period and determining a value of the second bias voltage in dependence upon the one or more target acoustic frequencies.

In other words, the method may include a step of tuning the bias voltage in accordance with a harmonic frequency to be measured. This may for example make use of a pre-defined mapping function or lookup table which relates target frequencies to optimal bias voltages for sampling those frequencies.

The method could further include a step of determining or identifying the one or more target acoustic frequencies to be measured, and wherein the one or more target acoustic frequencies are each harmonics of the frequency of the RF voltage applied during the transmit period. In other words, if the central transmit frequency is known (e.g. this may be identified from a register entry in a processor register), then the target acoustic frequencies can be determined as those frequencies which are pre-defined harmonics, e.g. first or second or third harmonics, of the central transmit frequency.

In some advantageous embodiments, the one or more target acoustic frequencies may include a third harmonic of the frequency of the RF voltage applied during the transmit period.

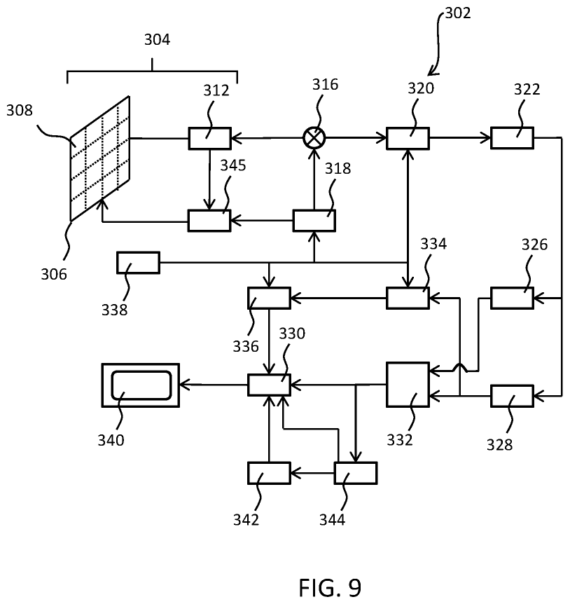

Certain embodiments employ use of drive electronics and/or receive or sampling electronics. By way of further, more detailed explanation, the general operation of an exemplary ultrasound imaging system which includes the drive electronics, receive/sampling electronics and also image forming components will now be described, with reference to FIG. 9.

The system 302 comprises an ultrasound probe, in particular an array transducer probe 304, which has a transducer array 306 for transmitting ultrasound waves and receiving echo information. The transducer array 306 comprises cMUT transducers. In this example, the transducer array 306 is a two-dimensional array of transducers 308 capable of scanning either a 2D plane or a three-dimensional volume of a region of interest. In another example, the transducer array may be a 1D array.

The transducer array 306 is coupled to a microbeamformer 312 which controls reception of signals by the transducer elements. Microbeamformers are capable of at least partial beamforming of the signals received by sub-arrays, generally referred to as “groups” or “patches”, of transducers as described in U.S. Pat. No. 5,997,479 (Savord et al.), U.S. Pat. No. 6,013,032 (Savord), and U.S. Pat. No. 6,623,432 (Powers et al.).

It should be noted that the microbeamformer is in general entirely optional. Further, the system includes a transmit/receive (T/R) switch 316, which the microbeamformer 312 can be coupled to and which switches the array between transmission and reception modes, and protects the main beamformer 320 from high energy transmit signals in the case where a microbeamformer is not used and the transducer array is operated directly by the main system beamformer. The transmission of ultrasound beams from the transducer array 306 is directed by a transducer controller 318 coupled to the microbeamformer by the T/R switch 316 and a main transmission beamformer (not shown), which can receive input from the user's operation of the user interface or control panel 338. The controller 318 can include transmission circuitry arranged to drive the transducer elements of the array 306 (either directly or via a microbeamformer) during the transmission mode.

The function of the control panel 338 in this example system may be facilitated by an ultrasound controller unit according to an embodiment of the invention.

In a typical line-by-line imaging sequence, the beamforming system within the probe may operate as follows. During transmission, the beamformer (which may be the microbeamformer or the main system beamformer depending upon the implementation) activates the transducer array, or a sub-aperture of the transducer array. The sub-aperture may be a one-dimensional line of transducers or a two dimensional patch of transducers within the larger array. In transmit mode, the focusing and steering of the ultrasound beam generated by the array, or a sub-aperture of the array, are controlled as described below.

Upon receiving the backscattered echo signals from the subject, the received signals undergo receive beamforming (as described below), in order to align the received signals, and, in the case where a sub-aperture is being used, the sub-aperture is then shifted, for example by one transducer element. The shifted sub-aperture is then activated and the process repeated until all of the transducer elements of the transducer array have been activated.

For each line (or sub-aperture), the total received signal, used to form an associated line of the final ultrasound image, will be a sum of the voltage signals measured by the transducer elements of the given sub-aperture during the receive period. The resulting line signals, following the beamforming process below, are typically referred to as radio frequency (RF) data. Each line signal (RF data set) generated by the various sub-apertures then undergoes additional processing to generate the lines of the final ultrasound image. The change in amplitude of the line signal with time will contribute to the change in brightness of the ultrasound image with depth, wherein a high amplitude peak will correspond to a bright pixel (or collection of pixels) in the final image. A peak appearing near the beginning of the line signal will represent an echo from a shallow structure, whereas peaks appearing progressively later in the line signal will represent echoes from structures at increasing depths within the subject.

One of the functions controlled by the transducer controller 318 is the direction in which beams are steered and focused. Beams may be steered straight ahead from (orthogonal to) the transducer array, or at different angles for a wider field of view. The steering and focusing of the transmit beam may be controlled as a function of transducer element actuation time.

Two methods can be distinguished in general ultrasound data acquisition: plane wave imaging and “beam steered” imaging. The two methods are distinguished by a presence of the beamforming in the transmission (“beam steered” imaging) and/or reception modes (plane wave imaging and “beam steered” imaging).

Looking first to the focusing function, by activating all of the transducer elements at the same time, the transducer array generates a plane wave that diverges as it travels through the subject. In this case, the beam of ultrasonic waves remains unfocused. By introducing a position dependent time delay to the activation of the transducers, it is possible to cause the wave front of the beam to converge at a desired point, referred to as the focal zone. The focal zone is defined as the point at which the lateral beam width is less than half the transmit beam width. In this way, the lateral resolution of the final ultrasound image is improved.

For example, if the time delay causes the transducer elements to activate in a series, beginning with the outermost elements and finishing at the central element(s) of the transducer array, a focal zone would be formed at a given distance away from the probe, in line with the central element(s). The distance of the focal zone from the probe will vary depending on the time delay between each subsequent round of transducer element activations. After the beam passes the focal zone, it will begin to diverge, forming the far field imaging region. It should be noted that for focal zones located close to the transducer array, the ultrasound beam will diverge quickly in the far field leading to beam width artifacts in the final image. Typically, the near field, located between the transducer array and the focal zone, shows little detail due to the large overlap in ultrasound beams. Thus, varying the location of the focal zone can lead to significant changes in the quality of the final image.

It should be noted that, in transmit mode, only one focus may be defined unless the ultrasound image is divided into multiple focal zones (each of which may have a different transmit focus).

In addition, upon receiving the echo signals from within the subject, it is possible to perform the inverse of the above described process in order to perform receive focusing. In other words, the incoming signals may be received by the transducer elements and subject to an electronic time delay before being passed into the system for signal processing. The simplest example of this is referred to as delay-and-sum beamforming. It is possible to dynamically adjust the receive focusing of the transducer array as a function of time.

Looking now to the function of beam steering, through the correct application of time delays to the transducer elements it is possible to impart a desired angle on the ultrasound beam as it leaves the transducer array. For example, by activating a transducer on a first side of the transducer array followed by the remaining transducers in a sequence ending at the opposite side of the array, the wave front of the beam will be angled toward the second side. The size of the steering angle relative to the normal of the transducer array is dependent on the size of the time delay between subsequent transducer element activations.

Further, it is possible to focus a steered beam, wherein the total time delay applied to each transducer element is a sum of both the focusing and steering time delays. In this case, the transducer array is referred to as a phased array.

To provide the DC bias voltage for the cMUT transducers, the transducer controller 118 can be coupled to control a DC bias control 345 for the transducer array. The DC bias control 345 sets DC bias voltage(s) that are applied to the cMUT transducer elements.

For each transducer element of the transducer array, analog ultrasound signals, typically referred to as channel data, enter the system by way of the reception channel. In the reception channel, partially beamformed signals are produced from the channel data by the microbeamformer 312 and are then passed to a main receive beamformer 320 where the partially beamformed signals from individual patches of transducers are combined into a fully beamformed signal, referred to as radio frequency (RF) data. The beamforming performed at each stage may be carried out as described above, or may include additional functions. For example, the main beamformer 320 may have 328 channels, each of which receives a partially beamformed signal from a patch of dozens or hundreds of transducer elements. In this way, the signals received by thousands of transducers of a transducer array can contribute efficiently to a single beamformed signal.

The beamformed reception signals are coupled to a signal processor 322. The signal processor 122 can process the received echo signals in various ways, such as: band-pass filtering; decimation; I and Q component separation; and harmonic signal separation, which acts to separate linear and nonlinear signals so as to enable the identification of nonlinear (higher harmonics of the fundamental frequency) echo signals returned from tissue and micro-bubbles. This facilitates for example harmonic imaging. The signal processor may also perform additional signal enhancement such as speckle reduction, signal compounding, and noise elimination. The band-pass filter in the signal processor can be a tracking filter, with its pass band sliding from a higher frequency band to a lower frequency band as echo signals are received from increasing depths, thereby rejecting noise at higher frequencies from greater depths that is typically devoid of anatomical information.

The beamformers for transmission and for reception are implemented in different hardware and can have different functions. Of course, the receiver beamformer is designed to take into account the characteristics of the transmission beamformer. In FIG. 9 only the receiver beamformers 312, 320 are shown, for simplicity. In the complete system, there will also be a transmission chain with a transmission micro beamformer, and a main transmission beamformer.

The function of the micro beamformer 312 is to provide an initial combination of signals in order to decrease the number of analog signal paths. This is typically performed in the analog domain.

The final beamforming is done in the main beamformer 320 and is typically after digitization.

The transmission and reception channels use the same transducer array 306 which has a fixed frequency band. However, the bandwidth that the transmission pulses occupy can vary depending on the transmission beamforming used. The reception channel can capture the whole transducer bandwidth (which is the classic approach) or, by using bandpass processing, it can extract only the bandwidth that contains the desired information (e.g. the harmonics of the main harmonic).

The RF signals may then be coupled to a B mode (i.e. brightness mode, or 2D imaging mode) processor 326 and a Doppler processor 328. The B mode processor 126 performs amplitude detection on the received ultrasound signal for the imaging of structures in the body, such as organ tissue and blood vessels. In the case of line-by-line imaging, each line (beam) is represented by an associated RF signal, the amplitude of which is used to generate a brightness value to be assigned to a pixel in the B mode image. The exact location of the pixel within the image is determined by the location of the associated amplitude measurement along the RF signal and the line (beam) number of the RF signal. B mode images of such structures may be formed in the harmonic or fundamental image mode, or a combination of both as described in U.S. Pat. No. 6,283,919 (Roundhill et al.) and U.S. Pat. No. 6,458,083 (Jago et al.) The Doppler processor 328 processes temporally distinct signals arising from tissue movement and blood flow for the detection of moving substances, such as the flow of blood cells in the image field. The Doppler processor 328 typically includes a wall filter with parameters set to pass or reject echoes returned from selected types of materials in the body.

The structural and motion signals produced by the B mode and Doppler processors are coupled to a scan converter 332 and a multi-planar reformatter 344. The scan converter 332 arranges the echo signals in the spatial relationship from which they were received in a desired image format. In other words, the scan converter acts to convert the RF data from a cylindrical coordinate system to a Cartesian coordinate system appropriate for displaying an ultrasound image on an image display 340. In the case of B mode imaging, the brightness of pixel at a given coordinate is proportional to the amplitude of the RF signal received from that location. For instance, the scan converter may arrange the echo signal into a two dimensional (2D) sector-shaped format, or a pyramidal three dimensional (3D) image. The scan converter can overlay a B mode structural image with colors corresponding to motion at points in the image field, where the Doppler-estimated velocities to produce a given color. The combined B mode structural image and color Doppler image depicts the motion of tissue and blood flow within the structural image field. The multi-planar reformatter will convert echoes that are received from points in a common plane in a volumetric region of the body into an ultrasound image of that plane, as described in U.S. Pat. No. 6,443,896 (Detmer). A volume renderer 342 converts the echo signals of a 3D data set into a projected 3D image as viewed from a given reference point as described in U.S. Pat. No. 6,530,885 (Entrekin et al.).

The 2D or 3D images are coupled from the scan converter 332, multi-planar reformatter 344, and volume renderer 342 to an image processor 330 for further enhancement, buffering and temporary storage for optional display on an image display 340. The imaging processor may be adapted to remove certain imaging artifacts from the final ultrasound image, such as: acoustic shadowing, for example caused by a strong attenuator or refraction; posterior enhancement, for example caused by a weak attenuator; reverberation artifacts, for example where highly reflective tissue interfaces are located in close proximity; and so on. In addition, the image processor may be adapted to handle certain speckle reduction functions, in order to improve the contrast of the final ultrasound image.

In addition to being used for imaging, the blood flow values produced by the Doppler processor 328 and tissue structure information produced by the B mode processor 126 are coupled to a quantification processor 334. The quantification processor produces measures of different flow conditions such as the volume rate of blood flow in addition to structural measurements such as the sizes of organs and gestational age. The quantification processor may receive input from the user control panel 338, such as the point in the anatomy of an image where a measurement is to be made.

Output data from the quantification processor is coupled to a graphics processor 336 for the reproduction of measurement graphics and values with the image on the display 340, and for audio output from the display device 340. The graphics processor 336 can also generate graphic overlays for display with the ultrasound images. These graphic overlays can contain standard identifying information such as patient name, date and time of the image, imaging parameters, and the like. For these purposes the graphics processor receives input from the user interface 338, such as patient name. The user interface is also coupled to the transmit controller 318 to control the generation of ultrasound signals from the transducer array 306 and hence the images produced by the transducer array and the ultrasound imaging system. The transmit control function of the controller 318 is only one of the functions performed. The controller 318 also takes account of the mode of operation (given by the user) and the corresponding required transmitter configuration and band-pass configuration in the receiver analog to digital converter. The controller 318 can be a state machine with fixed states.

The user interface is also coupled to the multi-planar reformatter 344 for selection and control of the planes of multiple multi-planar reformatted (MPR) images which may be used to perform quantified measures in the image field of the MPR images.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

A single processor or other unit may fulfill the functions of several items recited in the claims.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”.

Any reference signs in the claims should not be construed as limiting the scope.

1. A method for driving a cMUT device with a drive cycle comprising a transmit period and a receive period, the method comprising:
in the transmit period, driving a cMUT element of the cMUT device with a first bias voltage and an RF voltage;
in the receive period, driving the cMUT element with a second bias voltage, and without an RF voltage;
wherein the second bias voltage is higher than the first bias voltage; and
wherein the combined RF voltage and the first bias voltage are such as to cause the cMUT element to operate in a collapsed mode during the transmit period, and wherein the second bias voltage is such as to cause the cMUT element to operate in collapsed mode during the receive period;
wherein the difference between the second bias voltage and the first bias voltage is equal to a voltage amplitude of the RF voltage; and
wherein a sum of the voltage amplitude of the RF voltage and the first bias voltage is at or close to a maximum operational voltage.

2. (canceled)

3. The method of claim 1, or wherein the method further comprises sampling the cMUT element during the receive period to obtain a receive signal.

4. The method of claim 1,
wherein a transition from the transmit period to the receive period of the imaging cycle comprises a ramp-up of the first bias voltage to the second bias voltage, according to a first ramping function; and
wherein a transition from the receive period to the transmit period of the cycle comprises a ramp-down of the second bias voltage to the first bias voltage according to a second ramping function.

5. The method of claim 4, wherein the first and second ramping function are controllable.

6. The method of claim 4, wherein each of the first and second ramping functions is a smooth linear function.

7. The method of claim 4, any wherein the method further comprises sampling the cMUT element during the receive period to obtain a receive signal, and wherein said sampling comprises sampling only between the end of the ramp-up of the bias voltage and the beginning of the ramp-down of the bias voltage.

8. The method of claim 1, any further comprising
obtaining an indication of one or more target acoustic frequencies to be sampled during the receive period; and
determining a value of the second bias voltage in dependence upon the one or more target acoustic frequencies.

9. The method of claim 8, wherein the method comprises determining the one or more target acoustic frequencies, and wherein the one or more target acoustic frequencies are each harmonics of the frequency of the RF voltage applied during the transmit period.

10. The method of claim 9, wherein the one or more target acoustic frequencies include a third harmonic of the frequency of the RF voltage applied during the transmit period.

11. A cMUT apparatus, comprising:
a cMUT device comprising at least one cMUT element; and
drive electronics, adapted to drive the cMUT element with a drive cycle comprising a transmit period and a receive period, the drive electronics adapted to:
in the transmit period, drive the cMUT element with a first bias voltage and an RF voltage;
in the receive period, drive the cMUT element with a second bias voltage and without an RF voltage;
wherein the second bias voltage is higher than the first bias voltage; and
wherein the combined RF voltage and first bias voltage cause the cMUT element to operate in a collapsed mode during the transmit period, and wherein the second bias voltage causes the cMUT element to operate in collapsed mode during the receive period;
wherein the difference between the second bias voltage and the first bias voltage is equal to a voltage amplitude of the RF voltage; and
wherein a sum of the voltage amplitude of the RF voltage and the first bias voltage is at or close to a maximum operational voltage.

12. The apparatus of claim 11, further comprising signal sampling electronics adapted to sample the cMUT element during the receive period to obtain a receive signal.

13. The apparatus of claim 11,
wherein a transition from the transmit period to the receive period of the imaging cycle comprises a ramp-up of the first bias voltage to the second bias voltage, according to a first ramping function; and
wherein a transition from the receive period to the transmit period of the cycle comprises a ramp-down of the second bias voltage to the first bias voltage according to a second ramping function.

14. An ultrasound probe, comprising the cMUT apparatus of claim 11.

15. An ultrasound imaging system, comprising the ultrasound probe of claim 14.
